(19) United States
(12) Patent Application Publication
(10) Pub. No.: US 2026/0335152 A1
(43) Pub. Date: Oct. 29, 2026

(54) SANITARY NAPKINS WITH ANGLED WINGS

(71) Applicant: Maria Elvira Clark, Bristow, VA (US)

(72) Inventor: Maria Elvira Clark, Bristow, VA (US)

(21) Appl. No.: 19/341,673

(22) Filed: Sep. 26, 2025

Related U.S. Application Data
(60) Provisional application No. 63/796,622, filed on Apr. 29, 2025.

(51) Int. Cl. A61F 13/56; A61F 13/47

(52) U.S. Cl. CPC A61F 13/5616; A61F 2013/4708

(57) ABSTRACT

A sanitary napkin with an innovative wing configuration designed to enhance ergonomic fit, secure attachment, and functional reliability. The napkin includes a topsheet, backsheet, and absorbent layer, with multiple wing segments (16, 18, 20, 22, 24, 26, 28, 30) positioned along the lateral edges of the napkin. Outer wing segments are angled inwardly (22°) to align with undergarment seams, preventing material bunching and ensuring smooth folding. Inner wing segments are narrower and shorter, conforming to garment curvature for added comfort. Adhesive strips (32) beneath each wing enable secure attachment to the undergarment and overlapping wings, enhancing lateral stability during movement. Central wings may feature alternative shapes, such as truncated triangles or rectangles, to accommodate high-mobility activities. The napkin is available in various sizes, with wing spacing and adhesive placement tailored for optimal performance. This design provides improved comfort, protection, and adaptability for diverse user needs.

### CROSS-REFERENCE TO RELATED APPLICATION

This application claims the benefit of priority of U.S. provisional application number 63/796,622, filed Apr. 29, 2025, the contents of which are herein incorporated by reference.

### BACKGROUND OF THE INVENTION

The present invention relates to feminine hygiene products and, more particularly, to sanitary napkins with improved wing designs for enhanced attachment, comfort, and functionality.

The field of feminine hygiene products includes a wide range of absorbent articles designed to manage menstrual flow while providing comfort, discretion, and dependable performance. Among these, sanitary napkins have long been a commonly used solution for individuals seeking a non-invasive, easy-to-use option. Modern designs typically incorporate an absorbent layer, a fluid-permeable topsheet, and a backsheet equipped with adhesive to secure the napkin within an undergarment. In recent years, various configurations of side panels or “wings” have been introduced to improve lateral stability and reduce the likelihood of shifting or leakage. Despite these advancements, there remains considerable opportunity to refine how such wings interact with undergarment contours and adapt to users' movements.

Sanitary napkins aim to achieve several objectives: secure attachment to the undergarment, minimal disruption to wearer mobility, and prevention of fluid leakage. To accomplish these goals, manufacturers have experimented with wing shapes, adhesive patterns, and material stiffness. An optimal wing configuration would conform seamlessly to the natural curvature of the wearer's panty, maintain adherence over extended periods of activity, and avoid causing pressure points or chafing against sensitive skin. Additionally, wings are designed to resist adhesive-to-adhesive contact that compromises bond strength, and they are intended to allow for straightforward unfolding and refolding without tearing or wrinkling. Striking a balance between structural support and ergonomic flexibility is highly sought after in this market.

Conventional wing designs, however, exhibit a number of limitations that undermine these applicative goals. Many current products employ continuous or broad wing panels that are intended to lie flat against the undergarment but instead tend to bunch or form wrinkles when folded downward. These creases can inadvertently adhere to themselves, leading to partial or complete loss of adhesion to the panty and undermining secure placement. In addition, uniform wing widths fail to account for the tapering profile of most underwear crotch areas, resulting in uneven contact pressure and potential skin irritation along seam lines. Users engaging in everyday activities-or more strenuous exercise-may find that wings detach prematurely or restrict natural movement.

As can be seen, there is a need for sanitary napkins that more effectively conform to the natural contours of undergarments.

### SUMMARY OF THE INVENTION

In one aspect of the present invention, a sanitary napkin comprises a liquid-pervious topsheet, a liquid-impervious backsheet, and an absorbent layer positioned between the topsheet and backsheet. A plurality of discrete, non-contiguous wing segments is disposed along each lateral side of the napkin, each wing segment bearing a garment-facing adhesive. The wing segments include at least two outer segments and one inner segment per side, with adjacent segments spaced apart by a gap devoid of wing material. One outer segment is angled inwardly at an acute angle relative to the longitudinal centerline, and each wing segment is independently foldable about its own fold axis to adhere to an undergarment.

In embodiments, the wing segments comprise four segments per lateral side, each having a width of about 0.7 to 0.8 inches and a length of about 1.2 to 3.2 inches. The gap between adjacent segments measures about 0.5 to 1.75 inches. The garment-facing adhesive is provided as a strip with a length of about 1 to 1.5 inches and a width of about 0.5 to 1.25 inches. The outer or end wing segments are angled toward each end relative to the longitudinal centerline less than 90°, preferably less than 45°, such as about 20° to 24°. At least one inner wing segment has a truncated triangular shape, another may be rectangular with rounded corners, and the wing assembly may include first and second trapezoidal wings positioned centrally along the pad length. The napkin has a central width of about 2.4 to 2.6 inches. These and other features will be more fully understood from the following detailed description and accompanying drawings.

The wings incorporate angulation to match the upward curvature of modern panty designs, allowing corners to fold smoothly without compromising adhesive effectiveness. By tailoring wing geometry-both in terms of individual panel dimensions and angular orientation-manufacturers can provide a product that adheres more reliably, follows the undergarment's natural seams, and enhances wearer comfort during prolonged use.

These and other features, aspects and advantages of the present invention will become better understood with reference to the following drawings, description, and claims.

### BRIEF DESCRIPTION OF THE DRAWINGS

FIG. 1 is a front view of a panty liner with individual wings according to an embodiment of the present invention, shown in use;

FIG. 2 is a top perspective view thereof;

FIG. 3 is a bottom perspective view thereof;

FIG. 4 is a top plan view thereof, shown adhered to a panty with the individual wings in an extended position;

FIG. 5 is a bottom plan view thereof, shown adhered to a panty with the individual wings in an extended position;

FIGS. 6-7 are bottom perspective views thereof, illustrating sequential steps of securing the wings to a panty according to an embodiment of the present invention;

FIG. 8A is a top plan view of a panty liner with individual wings according to a second embodiment of the present invention;

FIG. 8B is a bottom plan view thereof;

FIG. 9A is a top plan view of a panty liner with individual wings according to a third embodiment of the present invention; and

FIG. 9B is a bottom plan view thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a sanitary napkin with a novel wing configuration that enhances both ergonomic fit and functional reliability. The sanitary napkin described herein has a generally hourglass shape and comprises a multi-layered structure designed to enhance comfort, absorbency, and convenience. The top sheet, made of a soft, non-woven fabric, e.g., polypropylene, provides a skin-friendly surface that facilitates rapid fluid transfer while maintaining a dry feel against the skin. Beneath the top sheet lies the transfer layer, which evenly distributes fluid across the absorbent section, preventing localized saturation and improving overall efficiency. The absorbent section generally consists of one to four layers, each tailored for specific functions: the uppermost layer quickly absorbs fluid, while subsequent layers, often incorporating superabsorbent polymers (SAP) such as sodium polyacrylate and softwood fluff pulp, retain and lock in moisture to prevent leakage. A waterproof barrier, typically made of polyethylene film, forms the bottommost layer, ensuring that no fluid escapes the napkin and protecting the user's clothing. For added convenience, the sanitary napkin includes an integrated disposal pouch, allowing for discreet and hygienic disposal after use. This combination of layers and features provides enhanced performance, comfort, and practicality for the user.

In an embodiment, the napkin incorporates eight slim, elongated wings-four on each side-strategically positioned along the lateral edges. The outer wings are angled at a 22-degree orientation to align seamlessly with the natural seams of the panty, which prevents wing material bunching and ensures the wings fold smoothly around the crotch area, adhering securely to the underside of the panty. The central wings are narrower and shorter, allowing them to conform to the panty's curvature without creating pressure points or wrinkles. The elongated wings are designed to overlap and adhere to one another on the underside of the panty, providing enhanced stability and preventing detachment during movement.

In an embodiment, the outer wings extend 3.0 inches in length, while the central wings are 1.25 inches in length. Each wing may be about 0.75 inches wide. Placement of the wings is generally as follows: the first wing is positioned 1.5 to 2.5 inches from the front edge of the pad, the second wing is 3.25 to 5.0 inches from the front edge, the third wing is 3.25 to 5.0 inches from the back edge, and the fourth wing is 1.5 to 3 inches from the back edge. The wings are spaced apart by intervals ranging from 0.5 to 1.75 inches (from an edge of one wing to an edge of an adjacent wing), which prevents wing material bunching and allows each wing to fold smoothly around the panty's curvature.

Adhesive strips are positioned beneath each wing, typically measuring 1.0 to 1.5 inch in length and 0.5 to 1.25 inches in width, enabling secure attachment to both the undergarment and overlapping wings.

In alternative embodiments designed for strenuous activities, the central wings are merged into a single wing with either a truncated triangle or a rectangle with smooth corners. The truncated triangle configuration features a narrow base of approximately 0.5 inches in width, a broader top side of 1.25 inches in width, and a length of 1.25 inches. The rectangle configuration offers a uniform width of 1.25 inches, rounded corners for comfort, and a length of 1.25 inches. Both configurations are designed to fold downward without bunching or wrinkling, allowing the wings to adhere securely to the underside of the panty and to each other, thereby preventing detachment during movement.

The trapezoidal wings are strategically positioned along the lateral edges of the napkin to conform seamlessly to the natural curvature of the undergarment, ensuring a secure and ergonomic fit. Adhesive strips positioned beneath each wing facilitate inter-wing adhesion, creating a cohesive structure that resists detachment and maintains the napkin's position even during high-mobility activities. These central wings are placed precisely in the center of the pad, typically at 4.5 to 6.5 inches from either edge, depending on pad size.

The pad may be produced in a variety of sizes, ranging from about 9.0-13.0 inches long and about 3.0 inches wide, with a central width of about 2.5 inches for all sizes. Adhesive strips placed longitudinally on the pad may range from 8.0 to 13.0 inches in length and may be 0.75 to 1.0 inch in width, tailored to pad size and positioned longitudinally along the bottom surface.

The strategic placement and spacing of wings and adhesive strips across all embodiments ensure a secure, ergonomic fit that adapts to the wearer's movements and undergarment contours, providing reliable comfort and protection. This innovative approach balances structural support and ergonomic flexibility, resulting in a sanitary napkin that is more secure, comfortable, and adaptable to the diverse needs of modern users.

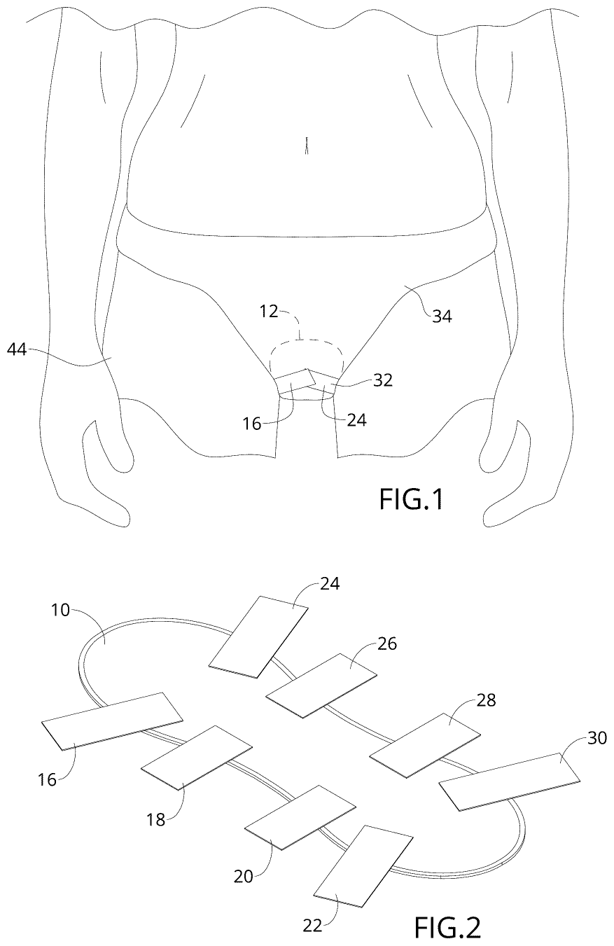

Referring to FIGS. 1-7, 8A, 8B, 9A, and 9B, FIG. 1 illustrates a sanitary napkin in use, with the bottom surface 12 interfacing with underwear 34 worn by the user 44. Wings 16, 24 are folded across the front surface of the underwear 34 and held in place by adhesive strips 32. While the wings in the drawings extend onto the top surface of the pad in the drawings, the present disclosure is not so limited. Preferably, the wings do not appear on the top surface of the pads.

Referring to FIGS. 2 and 4, the top surface 10 of the sanitary napkin is illustrated. The first wing 16, second wing 18, third wing 20, fourth wing 22, fifth wing 24, sixth wing 26, seventh wing 28, and eighth wing 30 are strategically positioned along the lateral edges of the sanitary napkin to ensure secure attachment and ergonomic fit. The wings are arranged symmetrically along the lateral edges, with the outer wings angled at about 22-23 degrees, denoted by the angle A, to align seamlessly with the natural seams of the underwear 34. This angular orientation ensures smooth folding and secure attachment, while the central wings provide additional stability by conforming to the curvature of the garment. The angled configuration of the wings prevents wing material bunching and enhances comfort during movement.

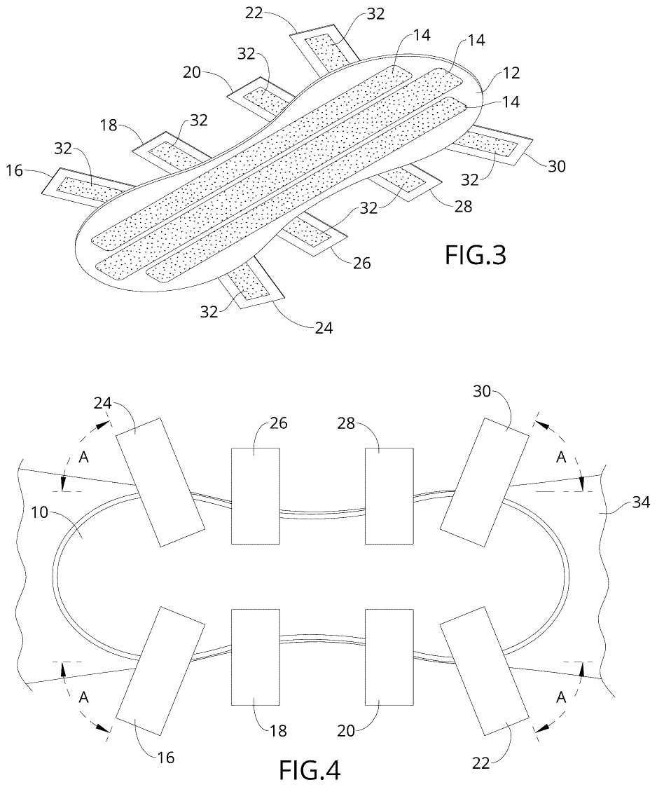

Referring to FIGS. 3 and 5, the bottom surface 12 of the sanitary napkin is equipped with adhesive strips 14 longitudinally positioned along the body of the napkin,. Adhesive strips 32 strategically placed beneath the first wing 16, second wing 18, third wing 20, fourth wing 22, fifth wing 24, sixth wing 26, seventh wing 28, and eighth wing 30 ensure reliable adherence to the underside of the underwear 34, even during high-mobility activities. The overlapping configuration of the wings, enabled by the adhesive strips, creates a cohesive structure that resists detachment and maintains the sanitary napkin's position.

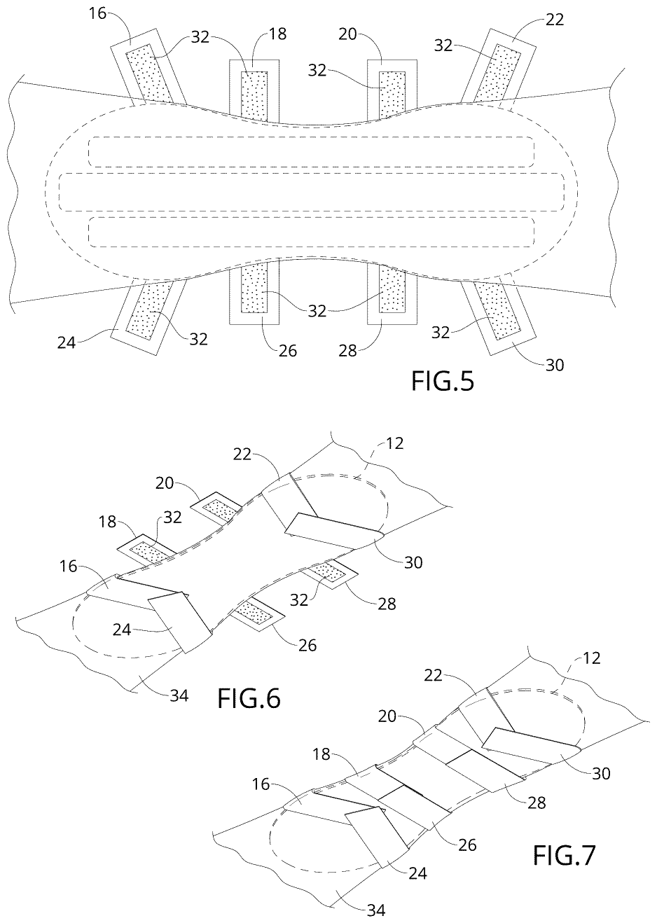

Referring to FIGS. 6 and 7, the bottom surface 12 of the sanitary napkin is shown in sequential perspective views, illustrating the overlapping folding and attachment progression of the first wing 16, fifth wing 24, fourth wing 22, and eighth wing 30, followed by the second wing 18, sixth wing 26, third wing 20, and seventh wing 28. These views demonstrate the functional interaction of the wings during use, highlighting their ability to conform to the garment's curvature and provide enhanced stability.

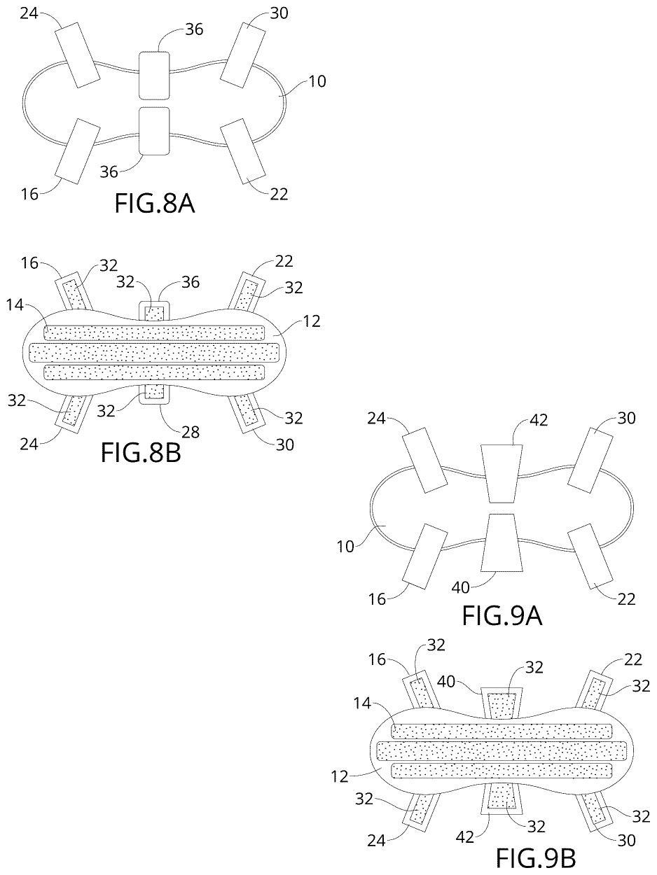

Referring to FIGS. 8A and 8B, the sanitary napkin according to the second embodiment is illustrated in top- and bottom-plan views. The top surface 10 forms the primary absorbent layer, while the bottom surface 12 provides the structural base for attachment. The sanitary napkin includes outer wings 16, 22, 24, 30, symmetrically positioned along the lateral edges and angled to align with the natural seams of the undergarment, preventing material bunching and ensuring smooth folding and secure adherence; and opposed rectangular wings 36 centrally positioned and providing additional stability. The bottom surface 12 is equipped with adhesive strips 14 running longitudinally along the bottom surface 12 and the wings 16, 22, 24, 30, 36 are equipped with adhesive strips 32, which are strategically positioned to ensure secure attachment to the undergarment. The adhesive system enables secure attachment to the underside of the panty and maintaining the napkin's position during high-mobility activities.

Referring to FIGS. 9A and 9B, the sanitary napkin according to the third embodiment is illustrated in top and bottom views, respectively. The top surface 10 forms the primary absorbent layer, while the bottom surface 12 provides the structural base for attachment. The sanitary napkin includes outer wings 16, 22, 24, 30, a first trapezoidal wing 40, and a second trapezoidal wing 42. The first trapezoidal wing 40 and the second trapezoidal wing 42 are centrally located and designed to conform to the curvature of the undergarment. Their geometry allows for smooth folding and secure adherence to the underside of the panty, cooperating with the other wings to provide enhanced stability and prevent detachment during high-mobility activities. These wings are strategically positioned along the lateral edges to ensure secure attachment and ergonomic fit. The arrangement, combined with the angled configuration, prevents material bunching and enhances comfort during movement.

The adhesive system comprises adhesive strips 14 running longitudinally along the bottom surface 12 and adhesive strips 32 positioned beneath each wing, including the first trapezoidal wing 40 and the second trapezoidal wing 42. The adhesive strips 14 provide secure attachment to the undergarment, while the adhesive strips 32 enable the wings to adhere to the underside of the panty and to each other, ensuring the sanitary napkin remains securely in place while maintaining comfort and protection for the user.

It should be understood, of course, that the foregoing relates to exemplary embodiments of the invention and that modifications may be made without departing from the spirit and scope of the invention as set forth in the following claims.

1. A sanitary napkin comprising:
a central pad including:
a liquid-pervious topsheet;
a liquid-impervious backsheet; and
an absorbent layer positioned between the liquid-pervious topsheet and the liquid-impervious backsheet; and
a plurality of discrete, non-contiguous wing segments disposed along each lateral side of the sanitary napkin, each wing segment bearing a garment-facing adhesive, the plurality comprising at least two outer wing segments and at least one inner wing segment on each lateral side, the at least one inner wing segment being disposed longitudinally between the at least two outer wing segments, the at least two outer wing segments on the same lateral side diverging from one another outward and away from a longitudinal centerline of the sanitary napkin, a first outer wing segment toward a front end of the central pad and a second outer wing segment toward a rear end of the central pad, adjacent wing segments on the same lateral side being spaced apart by a gap devoid of wing material;
wherein each wing segment is independently foldable about a respective fold axis to adhere, by the garment-facing adhesive borne by that wing segment, directly to an undergarment.

2. The sanitary napkin of claim 1, wherein the plurality of discrete, non-contiguous wing segments comprises four wing segments per lateral side.

3. The sanitary napkin of claim 1, wherein each wing segment has a width of about 0.7 to 0.8 inches and a length of about 1.2 to 3.2 inches.

4. The sanitary napkin of claim 1, wherein at least one inner wing segment has a truncated triangular shape.

5. (canceled)

6. The sanitary napkin of claim 1, wherein each garment-facing adhesive comprises an adhesive strip with: a length of about 1 to 1.5 inches; and a width of about 0.5 to 1.25 inches.

7. (canceled)

8. The sanitary napkin of claim 1, wherein at least one inner wing segment is a truncated triangular shape.

9. The sanitary napkin of claim 1, wherein at least one inner wing segment is rectangular with rounded corners.

10. The sanitary napkin of claim 1, wherein the sanitary napkin has a central width in a range of about 2.4 inches to about 2.6 inches.

11. The sanitary napkin of claim 1, wherein the wing segments comprise a first trapezoidal wing and a second trapezoidal wing each positioned centrally along a length of the central pad.

12. The sanitary napkin of claim 1, wherein each of the at least two outer wing segments on a lateral side is oriented at an acute angle relative to the at least one inner wing segment on that lateral side.

13. The sanitary napkin of claim 1, wherein at least one of the outer wing segments is oriented at an angle of about 20° to about 24° relative to the longitudinal centerline of the sanitary napkin.

14. The sanitary napkin of claim 1, wherein the gap between adjacent wing segments on the same lateral side is about 0.5 to about 1.75 inches.

15. The sanitary napkin of claim 1, wherein the central pad has a peripheral margin, the at least two outer wing segments are disposed along convex portions of the peripheral margin toward the front end and the rear end, and the at least one inner wing segment is disposed along a concave portion of the peripheral margin between the at least two outer wing segments.

16. The sanitary napkin of claim 1, wherein each lateral side comprises more than one inner wing segment, and the inner wing segments on that lateral side are parallel to one another.
